(19) United States
(12) Patent Application Publication
(10) Pub. No.: US 2008/0234765 A1
(43) Pub. Date: Sep. 25, 2008

(54) ROD REDUCTION METHODS AND DEVICES

(75) Inventors: William J. Frasier, New Bedford, MA (US); Paul G. Beaudoin, Derry, NH (US); John R. Cournoyer, Norfolk, MA (US); Michael S. Varieur, Portsmouth, RI (US); Dale E. Whipple, East Taunton, MA (US); Thomas J. Runco, Canton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 11/685,444

(22) Filed: Mar. 13, 2007

(51) Int. Cl. A61F 5/00; A61B 17/56; A61B 17/00

(52) U.S. Cl. 606/86 A; 606/208

(57) ABSTRACT

The present invention provides methods and devices for reducing a rod disposed within or adjacent to a rod-receiving head of a spinal implant. In general, a rod reduction device is provided that can removably mate to the rod-receiving head of a spinal implant and be effective to reduce a rod disposed within or adjacent to the rod-receiving head. In an exemplary embodiment, the rod reduction device can include a clamping member that is adapted to removably mate to a rod-receiving head of a spinal implant and a rod-reducing arm that is rotatably matable to the clamping member. The rod-reducing arm can be configured such that rotation of the arm with respect to the clamping member is effective to reduce a rod into the rod-receiving head of the spinal implant that is mated to the clamping member.

### FIELD OF THE INVENTION

The present invention relates to methods and devices for use in spinal surgery, and in particular to rod reduction devices and methods for using the same.

### BACKGROUND OF THE INVENTION

Spinal fixation devices are used in orthopedic surgery to align and/or fix a desired relationship between adjacent vertebral bodies. Such devices typically include a spinal connector, such as a relatively rigid fixation rod, that is coupled to adjacent vertebrae by attaching the element to various anchoring devices, such as hooks, bolts, wires, or screws. The fixation rods can have a predetermined contour that has been designed according to the properties of the target implantation site, and once installed, the instrument holds the vertebrae in a desired spatial relationship, either until desired healing or spinal fusion has taken place, or for some longer period of time.

Spinal fixation devices can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a rod-receiving element, usually in the form of a U-shaped slot formed in the head. A set-screw, plug, cap, or similar type of fastening mechanism, is used to lock the fixation rod into the rod-receiving head of the pedicle screw. In use, the shank portion of each screw is threaded into a vertebra, and once properly positioned, a fixation rod is seated through the rod-receiving member of each screw and the rod is locked in place by tightening a set screw or other fastener mechanism to securely interconnect each screw and the fixation rod.

While current spinal fixation systems have proven effective, one challenge associated with such systems is mounting the fixation rods into the rod-receiving member of various fixation devices. In particular, it can be difficult to align and seat the rod into the rod receiving portion of adjacent fixation devices due to the positioning and rigidity of the vertebra into which the fixation device is mounted. Thus, the use of a spinal rod reduction device, also sometimes referred to as a spinal rod approximator, is often required in order to grasp the head of the fixation device and reduce the rod into the rod-receiving head of the fixation device.

While several rod reduction devices are known in the art, some tend to be bulky and cumbersome to use. Accordingly, there is a need for improved rod reduction devices and methods for seating a spinal rod in a rod-receiving member of one or more spinal anchors.

### SUMMARY OF THE INVENTION

The present invention provides methods and devices for placing a spinal fixation rod into a rod-receiving head of a spinal implant. In one embodiment, the device can include a clamping member and a rod-reducing arm. The clamping member can be adapted to removably mate to a rod-receiving head of a spinal implant. The rod-reducing arm can be rotatably matable to the clamping member such that rotation of the rod-reducing arm with respect to the clamping member is effective to reduce a rod into a rod-receiving head of a spinal implant that is mated to the clamping member.

In general, the rod-reducing arm can include an elongate member having first and second opposed arms that are disposed at a distal end thereof. In one embodiment, the first and second opposed arms can be integrally formed on a distal end of the elongate member. In another embodiment, the first and second opposed arms can be pivotably coupled to a distal end of the elongate member. In this embodiment, the device can include an actuator that is operatively associated with the first and second opposed arms and is adapted to pivot the first and second opposed arms between open and closed positions.

In another embodiment, the rod-reducing arm can take the form of forceps that have first and second opposed arms that are movably coupled to one another at a location that is proximal to a distal end of each arm. A telescoping pin can extend between the first and second opposed arms and can be adapted to guide the first and second opposed arms between open and closed positions. The device can also include a ratcheting mechanism that is coupled to the first and second opposed arms and is adapted to lock the opposed arms in a fixed position.

The device can also include a post that extends outward from the rod-reducing arm and is adapted to abut against a rod to reduce a rod into a rod-receiving head of a spinal implant. In one embodiment, the post can include a concave distal surface that is adapted to engage a rod.

A variety of configurations are also available for the clamping member of the reduction device including, for example, first and second clamping jaws that are pivotably coupled to the first and second opposed arms of the rod-reducing arm and are adapted to removably mate to opposed sides of a rod-receiving head of a spinal implant. In one embodiment, the first and second clamping jaws are substantially C-shaped. A mating element can be formed on an inner facing surface of the first and second clamping jaws and be adapted to removably mate the jaws to a rod-receiving head of a spinal implant. The first and second clamping jaws can also be biased such that a force must be applied to the jaws to cause the jaws to pivot. In one embodiment, a pin can extend through the first and second clamping jaws and be adapted to maintain the alignment of the jaws with respect to each other and the opposed arms of the rod-reducing arm.

Exemplary rod reduction systems are also provided. In one embodiment, the rod reduction system can include a spinal implant and a rod reduction device. The spinal implant can include a rod-receiving head and a bone-engaging portion. A variety of configurations are available for the rod reduction device, including those discussed above. The system can further include a spinal rod that is adapted to be received by the rod-receiving head of the spinal implant.

Methods for reducing a rod into a rod-receiving head of a spinal implant are also provided. The method can generally include mating a clamping member of a rod reduction device to a rod-receiving head of a spinal implant and rotating a rod-reducing arm that is pivotally mated to the clamping member to thereby reduce a rod into the rod-receiving head of the spinal implant. In one embodiment, the clamping member can first be mated to the rod-receiving head of a spinal implant and the rod-reducing arm can then be mated to the clamping member to facilitate reduction of a rod into the rod-receiving head.

### BRIEF DESCRIPTION OF THE DRAWINGS

The invention will be more fully understood from the following detailed description taken in conjunction with the accompanying drawings, in which:

FIG. 1A is a perspective view of a rod reduction device according to one embodiment of the present invention;

FIG. 1B is another perspective view of the rod reduction device shown in FIG. 1A;

FIG. 1C is a perspective view of the rod reduction device shown in FIG. 1A mated to the rod-receiving head of a spinal implant;

FIG. 2A is a perspective view of another embodiment of a rod reduction device according to the present invention;

FIG. 2B is a perspective view of the rod reduction device shown in FIG. 2A mated to the rod-receiving head of a spinal implant;

FIG. 3A is a perspective view of another embodiment of a rod reduction device according to the present invention;

FIG. 3B is another perspective view of the rod reduction device shown in FIG. 3A;

FIG. 3C is another perspective view of the rod reduction device shown in FIG. 3A;

FIG. 3D is a perspective view of the rod reduction device shown in FIG. 3A mated to the rod-receiving head of a spinal implant;

FIG. 3E is another perspective view of the rod reduction device shown in FIG. 3A mated to the rod-receiving head of a spinal implant;

FIG. 4A is a perspective view of another embodiment of a rod reduction device according to the present invention;

FIG. 4B is another perspective view of the rod reduction device shown in FIG. 4A;

FIG. 5A is a perspective view of another embodiment of a rod reduction device according to the present invention showing the device mated to the rod-receiving head of a spinal implant and reducing a spinal rod; and

FIG. 5B is a side view of the rod reduction device shown in FIG. 5A.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein are methods and devices for reducing a rod disposed within or adjacent to a rod-receiving head of a spinal implant. In general, a rod reduction device is provided that can removably mate to the rod-receiving head of a spinal implant and be effective to reduce a rod disposed within or adjacent to the rod-receiving head. In an exemplary embodiment, the rod reduction device can include a clamping member that is adapted to removably mate to a rod-receiving head of a spinal implant and a rod-reducing arm that is rotatably matable to the clamping member. The rod-reducing arm can be configured such that rotation of the arm with respect to the clamping member is effective to reduce a rod into the rod-receiving head of the spinal implant that is mated to the clamping member. The rod reduction device can be used with a variety of spinal implants in addition to various other tools.

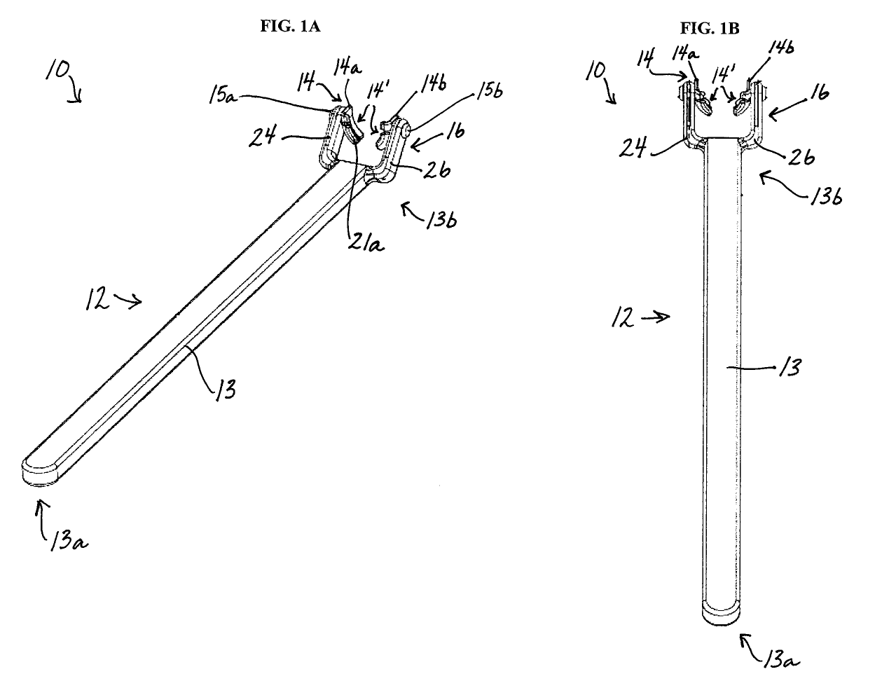

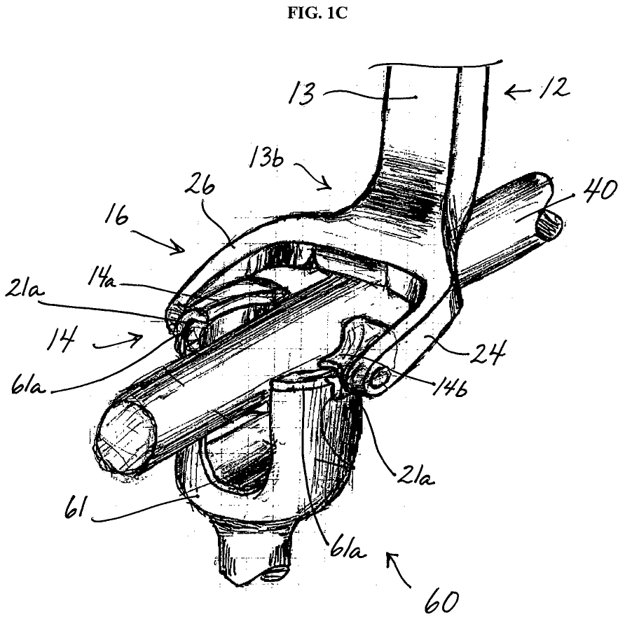

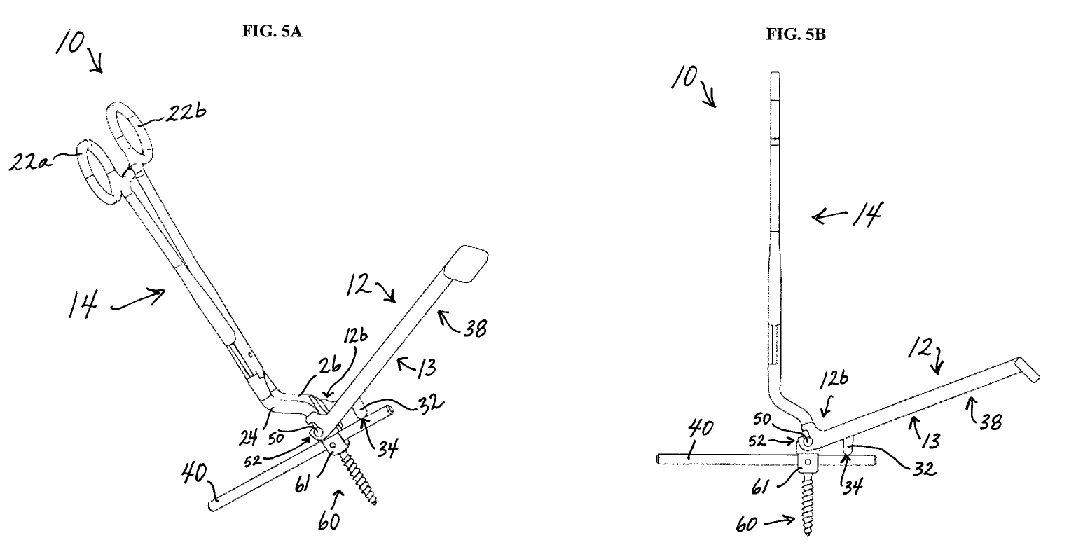

FIGS. 1A-5B illustrate exemplary embodiments of rod reduction devices 10. In general, each rod reduction device 10 disclosed herein includes a rod-reducing arm 12 that can be rotatably mated to a clamping member 14. Several configurations are available for the rod-reducing arm 12 and clamping member 14. In several embodiments (shown in FIGS. 1A-4B), the rod-reducing arm 12 and clamping member 14 can be mated prior to use such they form a single one-piece device 10. In another embodiment (shown in FIGS. 5A-5B), the rod-reducing arm 12 and clamping member 14 can be separate components that are mated to each other in use. In such an embodiment, the clamping member 14 can be first mated to a rod-receiving head of a spinal implant and the rod-reducing arm 12 can then be mated to the clamping member 14 to facilitate reduction of a rod into the rod-receiving head.

As indicated above, the rod reduction device 10 can include a rod-reducing arm 12. While a variety of configurations are available for the rod-reducing arm 12, the arm 12 can generally be an elongate member 13 that includes first and second opposed arms 24, 26 that are disposed at a distal end 13b thereof. In one exemplary embodiment, shown in FIGS. 1A-1C, the elongate member 13 is a rod that has a rectangular cross-section and a rounded proximal end 13a. In use, the surgeon can grasp a proximal portion 13a of the elongate member and slidably engage the rod-receiving head of a spinal implant with the distally disposed clamping member 14.

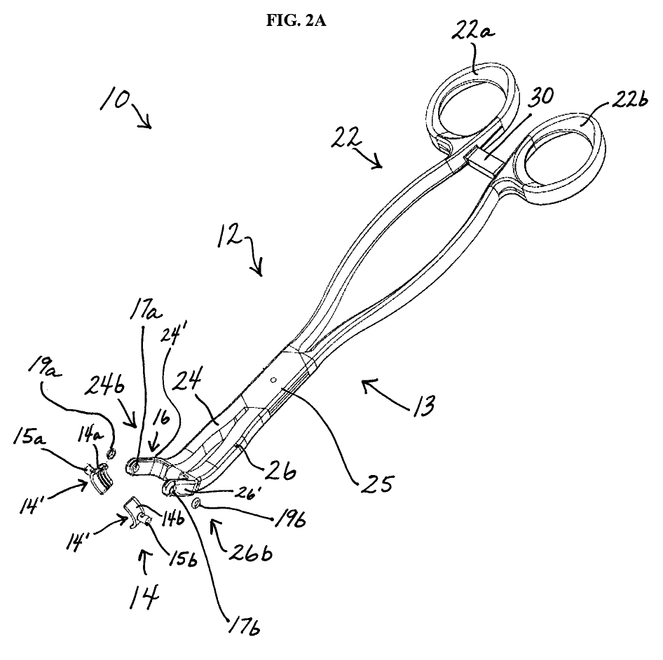

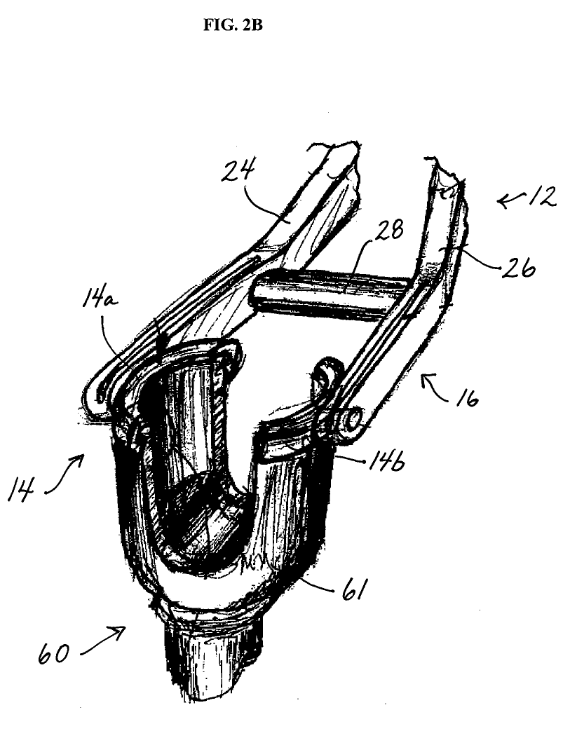

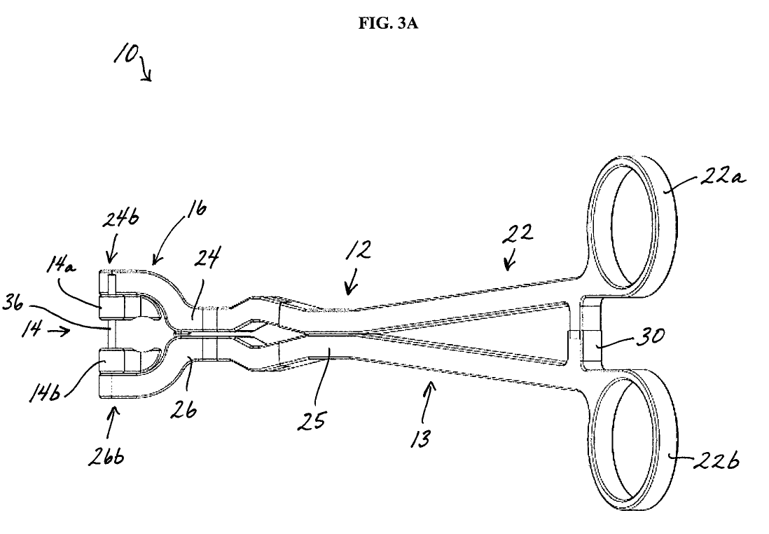

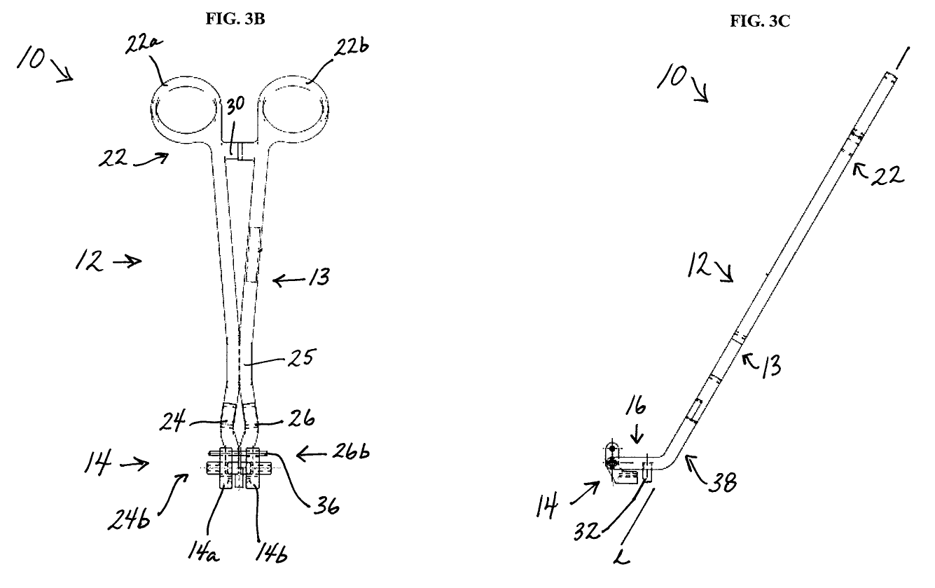

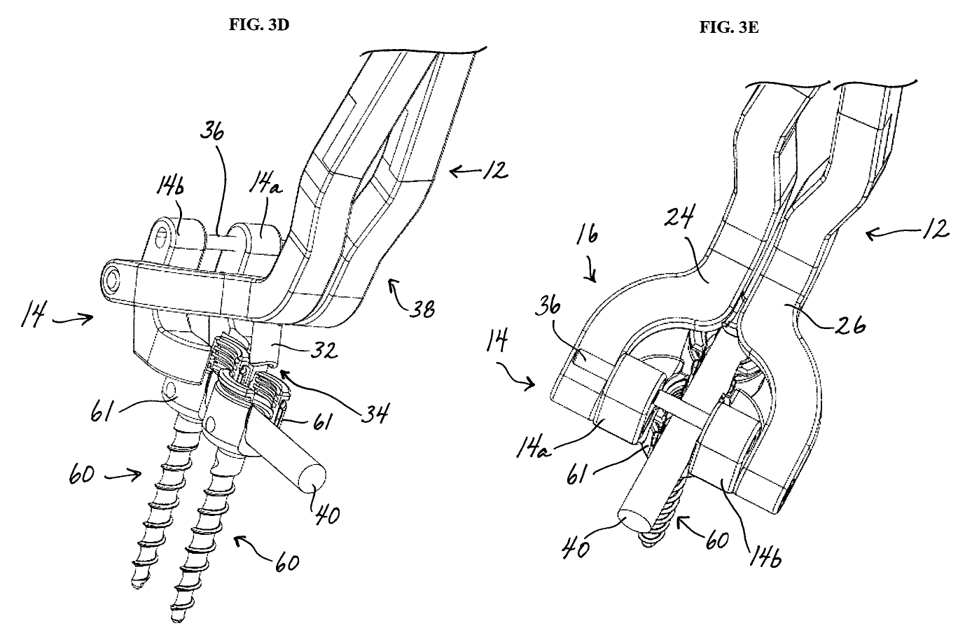

In another embodiment, the elongate member 13 can take the form of forceps. For example, as shown in FIGS. 2A-3E, the rod-reducing arm 12 resembles forceps with a handle portion 22 that is disposed at a proximal end 13a thereof and first and second opposed arms 24, 26 that extend distally from the handle portion 22. The handle portion 22 can include first and second finger loops 22a, 22b to facilitate grasping of the device 10. The first and second opposed arms 24, 26 can be movably coupled to one another at a location 25, such as a pivot, that is proximal to a distal end 24b, 26b of each arm 24, 26. In use, the handle portion 22 can be used to move the opposed arms 24, 26 from an open position, in which the device 10 can be positioned around the rod-receiving head of a spinal implant, to a closed position, in which the device 10 engages the rod-receiving head of the spinal implant. As shown in FIG. 2B, in one embodiment, a telescoping pin 28 extends between the first and second opposed arms 24, 26 and is adapted to guide the opposed arms 24, 26 between the open and closed positions. The device 10 can also include a ratcheting mechanism 30 (FIGS. 2A and 3A-3C) that is coupled to the first and second opposed arms 24, 26 and is adapted to lock the opposed arms 24, 26 in desired incremental positions.

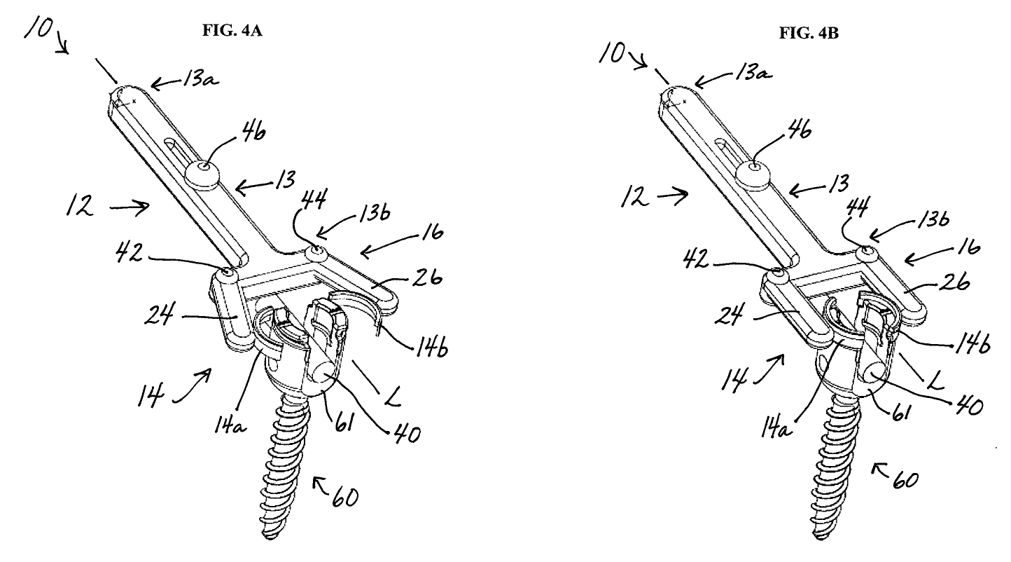

A variety of configurations are also available for the first and second opposed arms 24, 26 that are disposed at a distal end 13b of the elongate member 13 of the rod-reducing arm 12. In general, the opposed arms 24, 26 can form a substantially U-shaped member 16 at the distal end 12b of the elongate member 13. The U-shaped member 16 can be aligned with the longitudinal axis L of the elongate member 13 (as shown in FIGS. 4A-4B) or it can be offset from or extend from the elongate member 13 at an angle (as shown in FIGS. 1A-3E) to provide the rod-reducing arm 12 with additional leverage for reducing a rod. The first and second opposed arms 24, 26 can be formed integrally with the elongate member 13 or can be fixably or movably coupled thereto.

For example, in one exemplary embodiment, shown in FIGS. 4A-4B, the first and second opposed arms 24, 26 are pivotably coupled to a distal end 13b of the elongate member 13. As shown, the elongate member 13 is generally T-shaped and the first and second opposed arms 24, 26 are pivotably coupled a distal portion 13b of the elongate member 13 via pivot pins 42, 44. In this embodiment, an actuator knob 46 which connects to an actuator mechanism (not shown) can be disposed on the elongate member 13 and be operatively associated with the first and second opposed arms 24, 26. The actuator 46 can be adapted to pivot the first and second opposed arms 24, 26 between an open position, in which the device 10 can be positioned around the rod-receiving head of a spinal implant, to a closed position, in which the device 10 engages the rod-receiving head of the spinal implant.

The rod-reducing arm 12 of the rod-reduction device 10 can further include a reduction post 32 that is adapted to reduce a rod into a rod-receiving head of a spinal implant. In general, the reduction post 32 can extend outward from a bottom facing surface 38 of the rod-reducing arm 12 and be adapted to abut against a rod that is disposed adjacent to a rod-receiving head of a spinal implant that is mated to the clamping member 14 of the device 10. A variety of configurations are available for the reduction post 32. For example, as shown in FIGS. 3C-3D and 5A-5B, the post 32 is a cylindrical member that is mated to the rod-reducing arm 12. In another embodiment, the reduction post 32 can be formed integrally with the rod-reducing arm 12. As shown in FIG. 3D, the post 32 includes a concave distal surface 34 that is adapted to engage a rod. In use, the distal surface 34 of the post 32 can abut a spinal rod and apply a downward force to the rod as the rod-reducing arm is rotated with respect to the clamping member 14 of the device 10. The downward force can be effective to reduce the rod into the rod-receiving head of a spinal implant that is mated to the clamping member 14, or that is adjacent to the spinal implant that the clamping member is mated to. Although the reduction post 32 is shown and described as a cylindrical member having a concave distal surface 34, one skilled in the art will appreciate that the post 32 can take virtually any size and shape and include a distal surface 34 that is configured to mate with any shape spinal rod. In addition to being located on the post 32, the surface 34 that mates with the rod can be located on the surface of the elongate member 13 (FIGS. 1A-1C, 4A-4B), or on the surface of the telescoping pin 28 (FIG. 2B).

As indicated above, the rod reduction device 10 can also include a clamping member 14. The clamping member 14 of the rod reduction device 10 can have a variety of configurations and is generally adapted to securely grasp a rod-receiving head of a spinal implant. In one exemplary embodiment, shown in FIGS. 1A-1C, the clamping member 14 includes first and second clamping jaws 14a, 14b that are pivotably coupled to the first and second opposed arms 24, 26 of the rod-reducing arm 12 and are adapted to removably mate to opposed sides of a rod-receiving head of a spinal implant. The jaw members 14a, 14b are preferably positioned substantially parallel to, but spaced apart from, one another, as shown in FIG. 1B, to allow an implant (FIG. 1C) to be engaged therebetween. As shown, each jaw member 14a, 14b has a generally elongated, hemispherical shape and is pivotably coupled to an opposed arm 24, 26 of the rod-reducing arm 12 via a pivot pin 15a, 15b. The pivot pin 15a, 15b can be integrally formed with the jaw member 14a, 14b or can be a separate component that is mated thereto. While a variety of configurations are available for the pivot pin 15a, 15b, the pivot pin 15a, 15b can generally extend from the jaw member 14a, 14b and be received by a bore 17a, 17b formed in the opposed arm 24, 26. As shown in FIGS. 1A-2A, the pivot pin 15a, 15b extends through the bore 17a, 17b in the opposed arm 24, 26 and a washer 19a, 19b that is disposed on an outer surface 24′, 26′ of the opposed arm 24, 26 is provided to secure the pivot pin 15a, 15b thereto.

The clamping member 14 can be configured to maintain the parallel alignment of the first and second jaw members 14a, 14b with respect to each other and the opposed arms 24, 26 of the rod-reducing arm 12. A variety of techniques can be used to maintain the alignment of the jaw members 14a, 14b. For example, in one embodiment shown in FIGS. 3A-3E, a stationary pin 36 that extends through the first and second clamping jaws 14a, 14b is adapted to maintain the alignment of the jaws 14a, 14b. The clamping jaws 14a, 14b can be slidably engaged with the pin 36 such that the first and second jaw members 14a, 14b slide along the pin 36 as the first and second opposed arms 24, 26 are moved between open and closed positions. As the movement of the jaws 14a, 14b is restrained by the stationary pin 36, the pin 36 is thereby effective to maintain the alignment of the jaw members 14a, 14b with respect to each other and the opposed arms 24, 26.

In another embodiment, the jaw members 14a, 14b can be biased such that a force must be applied to the jaws 14a, 14b to cause the jaws 14a, 14b to pivot. Several techniques can be used to bias the clamping jaws 14a, 14b including, for example, biasing elements (not shown), such as springs, that are disposed in the first and second opposed arms 24, 26 of the rod-reducing arm 12. The springs can be positioned in a groove or channel formed in the first and second opposed arms 24, 26 and can extend from a proximal portion of the opposed arm 24, 26 to a distal end of the arm. Distal portions of the springs can be disposed in grooves formed in the pivot pins 15a, 15b that mate the jaw members 14a, 14b to the opposed arms 24, 26. In one exemplary embodiment, the springs can apply a downward force to the grooves formed in the pivot pins 15a, 15b to bias the jaw members 14a, 14b at an angle with respect to the opposed arms 24, 26. Such a configuration can maintain the parallel alignment of the of the jaw members 14a, 14b and prevent the jaw members 14a, 14b from pivoting out of alignment while the rod-reduction device 10 is being mated to a rod-receiving head of a spinal implant. In use, the springs can be configured to flex upward to allow the rod-reducing arm 12 to rotate with respect to the clamping jaws 14a, 14b to thereby reduce a rod into the rod-receiving head of the spinal implant that is engaged by the jaw members 14a, 14b. In addition to providing a biasing force, the springs can also facilitate attachment of the jaw members 14a, 14b to the opposed arms 24, 26.

The hemispherical surface 14′ that forms each jaw member 14a, 14b can be configured to mate to the rod-receiving head of a spinal implant. For example, in the exemplary embodiment shown in FIGS. 1A-1C, each jaw member 14a, 14b has a shape that is adapted to match the contour of the rod-receiving head of the spinal implant. As shown, the jaw members 14a, 14b taper toward one another to form a C-shape that is configured to fit around the rod-receiving head of an implant. Each jaw member 14a, 14b can also optionally include a mating element formed on an inner facing surface 14′ thereof that is effective to grasp the spinal implant. Virtually any mating element can be used, but in an exemplary embodiment, shown in FIGS. 1A and 1C, a channel 21a is formed on the inner facing surface 14′ of each jaw member 14a, 14b and it is adapted to engage a corresponding lip 61 a formed around the rod-receiving head 61 of a spinal implant 60. In use, the jaw members 14a, 14b can slidably engage the lip 61a formed on the rod-receiving head 61 to secure the rod-reduction device 10 to the spinal implant 60. In another embodiment (not shown), a pin member can be formed on the inner facing surface of each jaw member and it is adapted to be disposed within a corresponding detent formed in the rod-receiving head of a spinal implant. A person skilled in the art will appreciate that a variety of techniques can be used to mate the rod-reduction device 10 to the rod-receiving head 61 of a spinal implant 60.

As indicated above, in one exemplary embodiment, the rod-reducing arm 12 and clamping member 14 can be separate components that are mated to each other in use. Such a configuration is illustrated in FIGS. 5A-5B. The clamping member 14 can first be mated to a rod-receiving head of a spinal implant, and the rod-reducing arm 12 can then be mated to the clamping member 14 to facilitate reduction of a rod into the rod-receiving head. As shown, the clamping member 14 can take the form of forceps similar to those described above and shown in FIGS. 2A-3E. However, unlike the embodiments shown in FIGS. 2A-3E, the clamping member 14 shown in FIGS. 5A-5B does not include a reduction post disposed thereon. Instead, a rod reduction post 32 can be disposed on a rod-reducing arm 12 that is separate from the clamping member 14. As illustrated in FIGS. 5A-5B, the rod-reducing arm 12 is removably and rotatably coupled to the clamping member 14 to facilitate the reduction of the rod 40. The rod-reducing arm 12 can simply clamp onto the clamping member 14 or can include one or more mating elements that are adapted to mate to complementary formed mating elements on the clamping member 14 and/or the rod-receiving head of the spinal implant. In one exemplary embodiment, shown in FIGS. 5A-5B, the clamping member 14 includes a mating element in the form of a post 50 that is adapted to mate to a lip 52 that extends from a distal end 12b of the rod-reducing arm 12. One skilled in the art will appreciate that a variety of mating elements can be used to rotatably couple the rod-reducing arm 12 to the clamping member 14.

Methods for reducing a rod 40 into a rod-receiving head 61 of a spinal implant 60 are also provided. FIGS. 3D-3E illustrate one exemplary embodiment of the rod reduction device 10 in use with a spinal implant 60. The procedure first requires one or more spinal implants 60 to be secured within vertebral bone structures (not shown) during a surgical procedure. Typically, where two spinal implants 60 are fastened into adjacent vertebrae, a spinal rod 40 is inserted into the rod-receiving head 61 of each implant 60. Due to the alignment of the implants 60, however, it can be difficult to fully seat the rod 40 within each rod-receiving recess. Thus, the rod reduction device 10 can be used to reduce the spinal rod 40 into the rod-receiving heads 61 of the spinal implants 60.

As shown in FIGS. 3D-3E, the clamping member 14 of the rod reduction device 10 is mated to the rod-receiving head 61 of the spinal implant 60. In the embodiment shown, the reduction device 10 is mated to the rod-receiving head 61 by using the handle that is disposed on the proximal portion of the device 10 to move the first and second opposed arms 24, 26 of the rod reducing arm 12 to the open position. The clamping jaws 14a, 14b can then be positioned around the rod-receiving head 61 of the spinal implant 60 such that the spinal rod 40 extends between the first and second jaw members 14a, 14b. As explained above, the first and second jaw members 14a, 14b can be slidably disposed on the stationary pin 36 to maintain the alignment of the jaws 14a, 14b with respect to each other and the opposed arms 24, 26.

Once the clamping member 14 is properly positioned, the handle can be used to move the opposed arms to the closed position to cause the first and second jaw members 14a, 14b to engage the head 61 of the implant 60. A ratchet mechanism that is associated with the first and second opposed arms 24, 26 can be used to maintain the rod reduction device 10 in the closed position. The rod-reducing arm 12 can then be rotated with respect to the clamping member 14 to reduce the rod 40 into the rod-receiving head 61 of the spinal implant 60. As explained above, as the rod-reducing arm 12 is rotated, a reduction post 32 that is disposed on the arm 12 (or another portion of the arm) can abut the spinal rod 40 and apply a downward force to the rod 40 to reduce the rod 40 into the head 61 of the implant 60. When the rod 40 is fully reduced, the rod reduction device 10 can be removed from the spinal implant 60 and a fastener (not shown) can be applied to secure the rod within the head of the implant.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A rod reduction device, comprising:
a clamping member adapted to removably mate to a rod-receiving head of a spinal implant; and
a rod-reducing arm rotatably matable to the clamping member such that rotation of the rod-reducing arm with respect to the clamping member is effective to reduce a rod into a rod-receiving head of a spinal implant mated to the clamping member.

2. The device of claim 1, wherein the rod-reducing arm includes first and second opposed arms disposed at a distal end thereof.

3. The device of claim 2, wherein the clamping member comprises first and second clamping jaws pivotably coupled to the first and second opposed arms of the rod-reducing arm and adapted to removably mate to opposed sides of a rod-receiving head of a spinal implant.

4. The device of claim 3, wherein the first and second clamping jaws have a mating element formed on an inner facing surface thereof, the mating element being adapted to removably mate the jaws to a rod-receiving head of a spinal implant.

5. The device of claim 3, wherein the first and second clamping jaws are substantially C-shaped.

6. The device of claim 3, wherein the first and second clamping jaws are biased such that a force must be applied to the jaws to cause the jaws to pivot.

7. The device of claim 3, further comprising a pin extending through the first and second clamping jaws and adapted to maintain the alignment of the jaws with respect to each other and the opposed arms of the rod-reducing arm.

8. The device of claim 2, wherein the rod-reducing arm comprises an elongate member having the first and second opposed arms integrally formed on a distal end thereof.

9. The device of claim 2, wherein the rod-reducing arm comprises an elongate member having the first and second opposed arms pivotably coupled to a distal end thereof.

10. The device of claim 9, further comprising an actuator operatively associated with the first and second opposed arms and adapted to pivot the first and second opposed arms between open and closed positions.

11. The device of claim 1, wherein the rod-reducing arm comprises forceps having first and second opposed arms movably coupled to one another at a location proximal to a distal end of each arm.

12. The device of claim 11, further comprising a telescoping pin extending between the first and second opposed arms and adapted to guide the first and second opposed arms between open and closed positions.

13. The device of claim 1, further comprising a post extending outward from the rod-reducing arm and adapted to abut against a rod to reduce a rod into a rod-receiving head of a spinal implant mated to the clamping member.

14. The device of claim 13, wherein the post includes a concave distal surface adapted to engage a rod.

15. The device of claim 11, further comprising a ratcheting mechanism coupled to the first and second opposed arms and adapted to lock the opposed arms in a fixed position.

16. A rod reduction system, comprising:
a spinal implant having a rod-receiving head and a bone-engaging portion; and
a rod reduction device having a clamping member adapted to removably mate to the rod-receiving head of the spinal implant, and a rod-reducing arm rotatably matable to the clamping member such that rotation of the rod-reducing arm with respect to the clamping member is effective to reduce a rod into the rod-receiving head of the spinal implant when the clamping member is mated to the spinal implant.

17. The system of claim 16, further comprising a spinal rod adapted to be received by the rod-receiving head of the spinal implant.

18. The system of claim 16, wherein the rod-reducing arm includes first and second opposed arms disposed at a distal end.

19. The system of claim 18, wherein the clamping member comprises first and second clamping jaws pivotably coupled to the first and second opposed arms of the rod-reducing arm and adapted to removably mate to opposed sides of a rod-receiving head of a spinal implant.

20. A method for reducing a rod into a rod-receiving head of a spinal implant, comprising:
mating a clamping member of a rod-reduction device to a rod-receiving head of a spinal implant; and
rotating a rod-reducing arm pivotally mated to the clamping member to reduce a rod into the rod-receiving head of the spinal implant.

21. The method of claim 20, wherein the clamping member is first mated to a rod-receiving head of a spinal implant and the rod-reducing arm is then mated to the clamping member to facilitate reduction of a rod into the rod-receiving head.
